(19) United States
(12) Patent Application Publication
(10) Pub. No.: US 2024/0285060 A1
(43) Pub. Date: Aug. 29, 2024

(54) Coupled Magnetic System with Double-Adhesive for Eyelash Application

(71) Applicant: Keira Hsu, San Diego, CA (US)

(72) Inventor: Keira Hsu, San Diego, CA (US)

(21) Appl. No.: 18/421,887

(22) Filed: Jan. 24, 2024

Related U.S. Application Data
(63) Continuation-in-part of application No. 17/471,025, filed on Sep. 9, 2021.
(60) Provisional application No. 63/204,112, filed on Sep. 12, 2020.

(51) Int. Cl. A45D 40/30; A45D 2/48; A61K 8/19; A61K 8/22; A61K 8/46; A61Q 1/00

(52) U.S. Cl. CPC A45D 40/30; A45D 2/48; A61K 8/19; A61K 8/22; A61K 8/46; A61Q 1/00; A45D 2200/25; A61K 2800/88

(57) ABSTRACT

The present invention relates to an improvement in the field of lash lifts using a coupled magnetic system with double-adhesive tape for application of eyelash solutions. The coupled magnetic system comprises a base component for placement on an eyelid which attracts a band component to hold eyelashes inbetween the base and band components. In particular the coupled magnetic system allows for the application of formulations (permanent solutions and/or tint) to eyelashes without the concomitant use of liquid adhesive glue to the eyelashes.

### CROSS REFERENCE TO RELATED APPLICATIONS

This application is a continuation-in-part of U.S. application Ser. No. 17/471,025, filed Sep. 9, 2021, which in turn claims the benefit of U.S. Provisional Application No. 63/204,112, filed Sep. 12, 2020, each of which is incorporated herein by reference in their entireties.

### FIELD OF THE INVENTION

The present invention relates to an improvement in the field of lash lifts: a coupled magnetic system for application of eyelash solutions such as perm solutions or long-lasting tints. The coupled magnetic system comprises a base component for placement on a wearer's eyelid which attracts a band component to hold eyelashes inbetween the base and band components. In particular the coupled magnetic system allows for the application of formulations (permanent solutions and/or tint-dye solutions) to eyelashes without the concomitant use of adhesives to the eyelashes.

### BACKGROUND OF THE INVENTION

The field of lash lifts relates to the cosmetic method of applying solutions (permanent curling solutions or optionally dye tint solutions) to eyelashes, thereby causing the wearer's eyelashes to curl upwards in a more pronounced fashion or alternately to dye the wearer's eyelashes a darker, more pronounced color. The application of permanent solutions to eyelashes in a held fixed position, in a curl, provides a benefit of lasting up to six to eight weeks, for up to the life of the eyelashes, with minimum if any maintenance or upkeep. Moreover, having the eyelashes as curled onto a base provides a more natural upwards angle, rather than the angled crimping associated with a classic metal eyelash curler. Rod members for eyelash perms are described in the literature as in U.S. Pat. No. 6,536,445 and US2007012330.

Nonetheless, the current lash lift method of applying solutions to the eyelashes suffers from the need to apply a liquid adhesive to the eyelashes so as to adhere the eyelashes to the base pad. The need for a liquid adhesive step involves the application of an adhesive glue. This adhesive step often provides inconsistent results: the liquid adhesive requires time to achieve sufficient tackiness to adhere the eyelashes to the base, if not the eyelashes will stray, rendering the method ineffective. Oftentimes upon completion of the lash lift process the adhesive must then be removed from the eyelashes, sometimes resulting in clumping or worse, in unintentional loss of eyelashes. The liquid adhesive itself may also interfere in the permanent and dye tinting reactions such that the desired curling or tinting is not achieved. Moreover, many adhesives include ingredients which can cause allergic reaction, i.e. latex or formaldehyde.

The present invention is directed to a coupled magnetic system which comprises a base component having a first magnetic element and a band component having a second magnetic element, wherein the base component is placed on the surface of a wearer's eyelid, and the band component traps a wearer's eyelashes to the surface of the base component, without the need for an adhesive to be applied concomitantly to the eyelashes. The base component is placed on a wearer's eyelid and can optionally vary in length at specified ranges and optionally have a rounded edge to fit along the wearer's eyelid. The present invention further comprises a solution which can effectively react with a wearer's eyelashes, such as a perming solution or a dye-tint solution. The solution can optionally further include a thickening agent to allow for temporary adherence of the eyelash to the base component which the band component is applied.

The coupled magnetic system does not include a liquid adhesive for concomitant application to the eyelashes. In one embodiment, the coupled magnetic system further comprises a double adhesive tape applied on the bottom surface of the base component for adherence to a wearer's eyelid.

The present invention further comprises methods of using a coupled magnetic system to apply solutions to eyelashes comprising the following steps:
(a) placement of a base component having a first magnetic element on the surface of a wearer's eyelid;
(b) application of a solution to the wearer's eyelashes; and
(c) placement of a band component having a corresponding second magnetic element on top of the wearer's eyelashes such that the base component traps the eyelashes to the surface of the base component for a duration to allow the solution to effectively react with the wearer's eyelashes;
   wherein the method does not comprise the step of applying a liquid adhesive directly to the eyelashes. The methods of the present invention can be used for eyelash solutions such as permanent solutions or dye tinting solutions. The wearer's eyelashes can be brushed with solutions such that the eyelashes are trapped by the band component to the surface of the base component. The solutions of the present invention can include thickening or gelling agents to allow for temporary adherence (i.e. less than minute) of the eyelash solution to the base component to allow for placement of the band component, provided such thickening or gelling agent does not comprise adhesive. The base or band can each comprise magnetic elements which will attract the corresponding magnetic component.

The present invention is also directed to kits comprising a coupled magnetic system, as well as perming and eye tint solutions as described above.

Current lash systems for self-application on the market can be exemplified by Diablo Lash Lift sold by Diablo Cosmetics.

All references, publications, and patent applications disclosed herein are hereby incorporated by reference in their entirety.

### SUMMARY OF THE INVENTION

The present invention provides a coupled magnetic system for application of solutions to a wearer's eyelashes, the coupled magnetic system comprising a base component and a band component in which each comprise magnetic elements but does not include the application of an adhesive to the eyelashes.

The term “liquid adhesive” refers to agents which harden from liquid form into solid form, i.e. glue. Typically the adhesives which are avoided in the current invention are polymerizing agents such as acrylates, including amp-acrylates copolymer, ethyl acrylate or butyl acrylate, wherein the adhesive is intended to adhere the eyelashes to a base pad for at least eight to twelve minutes.

The term “double-adhesive tape” as directed to the current invention refer to tape having adhesive on both sides of the tape. Double-adhesive tapes of the current invention include, but are not limited to, Transpore Clear Plastic Tape by 3M, Micropore Tape by 3M, 3M double-sided silicone tape (96042) or nano tape or an equivalent thereof. Methods of making nano tape are disclosed U.S. Pat. No. 8,974,904 (Liming Dai), which is incorporated by reference in its entirety herein. Nano tape is sold alternately as nano tape, ‘alien’ tape or gecko tape. Nano tape are synthetic adhesive tapes consisting of arrays of carbon nanotubes transferred onto a backing material of flexible polymer tape.

The term “coupled magnetic system” refers to a system having two components, a base component which is placed on a wearer's eyelid, and a band component which is attracted to the base by magnetic attraction.

The term “magnetic elements” of the invention refers to either magnetic material or a metallic material to which a magnet is attracted, and can be any suitable magnetizable material comprising nickel, iron (including ferrous powder), cobalt, neodyminum or an alloy thereof (including steel or samarium-cobalt), or any polyvinyl resin comprising a magnetizable material such as by extrusion through a die, molding etc and can comprise, but is not limited to, a flexible rubber or silicone. The magnetic element can further be comprised of a magnetizable material such as magnetite or any other ferrous material which is magnetized after application to the substrate, and periodically re-magnetized. The magnetic particles can be magnetized in any desired direction by conventional means. Because magnetic force will penetrate the substrate surface of the base, when smaller individual magnetic elements are used they may be attached to either the top surface of the base component, or alternately to the bottom strip of the band, or both, so long as the base component attracts and holds the band component with eyelashes in between.

The base component can be made from any suitable material comprising at least one magnetic element for positioning on a wearer's eyelid. In one embodiment the coupled magnetic system, including both base component and band component, is prepared from a mixture of ferrite powder and rubber polymer resin. As illustrated in FIG. 3A, the base component can be a contoured flexible base comprising a first magnetic element and can vary in span from about 25 cm to about 40 cm to cover a wearer's eyelid; in one embodiment the span of the base can be about any of 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm or 45 mm. In a preferred embodiment, the span of the base component ranges from about 26 cm to about 38 cm. In one embodiment the base component design shape is widest at the span, such that the span aligns along the wearer's eyelid (see FIG. 4). As also illustrated in FIG. 3A, one embodiment of the present invention has a height of curvature (h) of the base component in a range from about 5 mm to about 11 mm for an optimal fit; in one embodiment the height of curvature (h) of the base component can be about any of 5 mm, 6 mm, 7 mm, 8 mm, 9 mm, 10 mm or 11 mm. In a preferred embodiment the height of the curvature (h) of the base component is about any of 6 mm, 7 mm, 8 mm, 9 mm or 10 mm. In a further embodiment, the thickness of base component has a maximum thickness of about 6 mm. The thickness of the base component can vary, depending on the desired curl of the wearer's eyelashes. In some embodiments, the thickness (t) of the base component is a maximum of about any of 1 mm, 2 mm, 3 mm or 4 mm.

The base component can be optionally further coated with a magnetic element on its surface, or alternately the base component can be completely comprised of a magnetic element or alternately have magnetic elements dispersed within the base. FIG. 1 illustrates a coupled magnetic system in which the base component is coated with a magnetic element, while the band component is consists essentially of a corresponding second magnetic element. FIG. 2 illustrates a coupled magnetic system in which the base component has two magnetic elements spaced apart, while the band component has two corresponding second magnetic elements that correspond in distance to the first magnetic elements in the base component.

The band component can be made from any suitable material comprising at least one corresponding second magnetic element which will attract the first magnetic element in the base component and hold or trap the wearer's eyelashes in-between, preferably on the surface of the base component. In one embodiment, the band component has a span that corresponds to the span of the base component. As described above, the band component can vary in span but optionally has a corresponding curvature and shape to fit the surface of the base component. In one embodiment the span of the band component matches the span of the base component, from about 25 cm to about 40 cm to trap the wearer's eyelashes; in some embodiments the span (s) of the band component can be about any of 25 cm, 26 cm, 27 cm, 28 cm, 29 cm, 30 cm, 31 cm, 32 cm, 33 cm, 34 cm, 35 cm, 36 cm, 37 cm, 38 cm, 39 cm or 40 cm. In a preferred embodiment, the span of the base component ranges from about 26 cm to about 38 cm. In a further embodiment, the height of curvature of the band component corresponds to match the height of curvature (h) of the base component. As shown in FIG. 3A, one embodiment of the present invention has a height of curvature (h) of the band component of about 5 mm to about 11 mm for an optimal fit; in one embodiment the height of curvature (h) of the band component can be about any of 5 mm, 6 mm, 7 mm, 8 mm, 9 mm, 10 mm or 11 mm. In a preferred embodiment the height of the curvature (h) of the band component is about any of 6 mm, 7 mm, 8 mm, 9 mm or 10 mm.

The present invention further describes a maximum thickness (t) of the base component. The thickness (t) of the base component impacts the upward curve of the wearer's eyelashes. In one embodiment, the thickness (t) of the component has a maximum thickness of about 7 mm. The thickness of the base component can vary depending on the desired upward angle of the wearer's eyelashes. In some embodiments, the thickness of the base component about any of 1 mm, 2 mm, 3 mm, 4 mm, 5 mm or 6 mm.

Embodiments of the coupled magnetic system would have adequate gauss to trap the wearer's eyelashes between the base and band components and furthermore to allow for easy removal of the band component, such as 200 gauss to 2000 gauss. An adequate gauss for the coupled magnetic systems described herein include 200 gauss, 300 gauss, 400 gauss, 500 gauss, 600 gauss, 700 gauss, 800 gauss, 900 gauss, 1000 gauss, 1100 gauss, 1200 gauss, 1300 gauss, 1400 gauss, 1500 gauss, 1600 gauss, 1700 gauss, 1800 gauss, 1900 gauss, 2000 gauss or ranges within.

To produce a component comprising a magnetic element, small uniform sized particles of the material can be incorporated into the rubber and/or siliconized mold or alternately into a polymer coating such as nylon-6-polymer or nitrocellulose, any of which allows the magnetic field to penetrate beyond the substrate. After the particles have been incorporated into the coating, the coating is then cured. Then the coating can be applied permanently to the substrate. Similar to a polymer coating, a plastic material can be embedded with such magnetic elements, or to smaller individual magnetic elements such as round neodymium magnets (roughly 1 mm, 2 mm, 3 mm to 4 mm in diameter) through methods known in the art.

In corresponding fashion, the corresponding second magnetic element can comprise one or more magnetic elements of opposing magnetic polarity, or can include metallic elements, any of which can attract the first magnetic element(s). In one embodiment, the coupled magnetic system comprises base and band components with first and second magnetic elements which share the same magnetic poles.

In one embodiment the corresponding second magnetic elements are attached towards the bottom edge of the base component and band component. The metallic elements are composed of a material that is magnetically attractive, including any of the magnetic metals described above, including iron, nickel, cobalt, neodymium, samarium-cobalt or alloys thereof so as to engage the corresponding second magnetic elements of the base component. If the magnetic material is located at discrete locations along the base components of the base component, then the metallic elements can be placed at corresponding locations of the bottom surface of the band component. Alternately the metallic element can coat the entire surface of the base component.

As the base component and band component are moved closer together, magnetic force between the magnetic and metallic elements in the assemblies cooperatively engage the two components with the wearer's eyelash sandwiched between the two components. The magnetic force is sufficient to keep the two components cooperatively engaged for the time required for the solutions to react with the keratin in the eyelashes (i.e. less than 5 minutes, less than 10 minutes, less than 15 minutes). The band component can then be lifted from the wearer's eyelashes. Each of the base component and band components can then be washed, sterilized or stored for further usage.

The present invention comprises a coupled magnetic system for the application of solutions to a wearer's eyelashes which comprises a base component having a first magnetic element and a band component having a second magnetic element, wherein the base component is placed on a wearer's eyelid and a solution is applied to the wearer's eyelashes, and the band component traps a wearer's eyelashes to the surface of the base component, without the need for a liquid adhesive glue to be applied concomitantly to the eyelashes.

The present invention achieves the objective of avoiding adhesive application to the eyelashes, and is fully reusable, easy to apply and self-align without the concomitant use of liquid adhesive glue.

The present invention further includes a method of using a coupled magnetic system to apply solutions to eyelashes comprising the following steps:
(a) placement of a base component having a first magnetic element on the surface of a wearer's eyelid;
(b) application of a solution to the wearer's eyelashes; and
(c) placement of a band component having a corresponding second magnetic element on top of the wearer's eyelashes such that the base component traps the eyelashes to the surface of the base component for a duration to allow the solution to effectively react with the wearer's eyelashes;
   wherein the method does not comprise the step of applying a liquid adhesive (in other words adhesive glue) directly to the eyelashes.

In the methods of the present invention, the solution can be a permanent (“perming”) solution for curling a wearer's eyelashes, including but not limited to a step 1 perming solution which comprises a reducing agent such as ammonium thioglycolate or a step 2 setting solution comprising an oxidizing agent such as hydrogen peroxide or sodium bromate. A step 1 perming solution can include any agent which reduces the disfulide bonds of a wearer's eyelashes, including but not limited to ammonium thioglycolate. The thioglycolate can be present in a weight % amount of 5%, 6%, 7%, 8%, 9%, 10%, 11%, 12%, 13%, 14%, 15%, 16%, 17%, 18%, 19% or 20% or in any range therein. In one embodiment of a step 1 perming solution, the solution comprises a weight % range of 7% to 15%, or a more preferred range of 9% to 12% of thioglycolate. In another embodiment of a step 1 perming solution, the step 1 perming solution further comprises a thickening agent such as mineral oil or hydrolyzed collagen. Another solution used for perming or curling a wearer's eyelashes is the second step (alternately, “step 2”) setting solution, which can be applied after the step 1 perming solution and comprises an oxidizing agent such as a diluted hydrogen peroxide or sodium bromate. The step 2 setting solution comprises an oxidizing agent selected from, but not limited to, sodium bromate or hydrogen peroxide in a weight % amount of 2%, 3%, 4%, 5%, 6%, 7%, 8%, 9%, 10%, 11%, 12%, 13% or 15%, or in any range therein. In one embodiment of a step 2 setting solution, the solution comprises a weight % range of 3% to 8% of oxidizing agent. The amount of time required to effectuate reaction of the solutions of the invention can be less than 5 minutes, less than 10 minutes or less than 15 minutes. Preferably the solutions when wetted to the lashes will cause the lashes to temporarily adhere to the base component, so as to facilitate the attraction of the band component. Optionally the solutions will further comprise thickening or gelling components to enhance temporary adhesion (i.e. less than a minute), without the use of a liquid adhesive agent. The thickening or gelling components of the solution do not include any adhesive components which would otherwise cause clumping or hardening, and if present would comprise less than 10% and more preferably less than 5% by weight of the overall applied solution. Preferably the thickening or gelling components contribute to a solution by using a combination of weak inter-molecular forces like hydrogen bonds, electrostatic forces, Van der Waals forces. Preferably any thickening or gelling components which are optionally included in the solution are hypoallergenic. Embodiments of thickening or gelling agents include mineral oil, hydrolyzed collagen or alternately, water-soluble, transparent colloids such as cellulose derivatives (i.e. hydroxycellulose), xanthan gums, or guar (i.e. boric acid) in trace amounts, i.e. about 1% to 10%. Thickening and gelling agents are described in the art, and are incorporated by reference. Current Developments in Excipient Science. R. Tekade, Advances in Pharmaceutical Product Development and Research. 2019, 29-83. I. W. Sutherland, Analysis of Glycan; Comprehensive Glycoscience, 2007. M-P Labeau, Guar and Guar Derivatives, Carbohydrate-based Polymer Building Blocks and Biopolymers, 2012 Elsevier.

The present inventions of the invention are preferably comprised of light colors or a transparent silver or white such that a wearer's eyelashes are clearly visible when trapped between the base component and band component. In one embodiment of the invention, both the base component and band component are painted white. In another embodiment of the invention the rubberized or siliconized base component is primarily transparent, save for the dispersion of magnetic elements.

In one embodiment of the invention, the coupled magnetic system is prepared from a dry mixture of ferrite powder and rubber polymer resin, which is mixed, calendered and ground, and then formed by either extrusion or rollers. In a further embodiment, the coupled magnetic system is prepared from a dry mixture of ferrite powder and rubber polymer resin, and then coated with white vinyl. Methods for preparing flexible magnets from ferrite powder and rubber polymer resin are well known in the art.

In one embodiment of the invention, the coupled magnetic system is prepared from a flexible magnetic sheet and the base component and band component are cut such that they have the same magnetic poles. The matching of magnetic poles enables the base component to precisely align the band component along the widest span of the base component, along the eyelid.

The present invention is further directed to kits comprising a coupled magnetic system as described above, and further comprising solutions for applications to the eyelashes. As described above, the solutions can comprise solutions useful for perming, including but limited to thioglycolate, hydrogen peroxide, or for dye-tinting. The kits of the invention can further comprise all elements useful for eyelash kits, including but not limited to eyelash brushes, pads for placement below the eye, or optionaling conditioning solutions or cleansing solutions.

The present invention is further directed to an applicator comprising a coupled magnetic system for the application of solutions in which the applicator comprises a fixed jaw as its base component and a moveable jaw as its band component, in which the base component has a first magnetic element and the band component has a second magnetic element. In one embodiment of the invention, the applicator is adapted from an eyelash curler known in the art, such as U.S. Pat. No. 2,602,458 (“Tuttle” incorporated by reference herein), which would allow for a user to apply the solutions of the invention in an aligned fashion on his or her own. Other eyelash curlers such as Japonesque® foldable eyelash curlers can be adapted to comprise a coupled magnetic system with a fixed jaw and moveable jaw each having magnetic elements as described above.

In an alternative embodiment of the present invention, the solution can comprise a dye tint solution, including but not limited to solutions having black carbon or charcoal.

Preferably the base component or band components are comprised of a clear or a light color so that the eyelashes can be visually distinguished when solutions are brushed on top.

The benefit of omitting a liquid adhesive application step also allows for solutions to penetrate the keratin of the eyelashes and to expedite faster and more convenient application of a solution to effectuate reaction, i.e. curling or dye of the eyelashes.

In one embodiment, the coupled magnetic system further comprises a double-adhesive tape applied to the bottom surface of the base component. In further embodiments of the current invention, the double-adhesive tape is nano tape. The benefit of a double adhesive tape as applied to the bottom surface of the base component allows for precision positioning on a wearer's eyelid.

### BRIEF DESCRIPTION OF THE DRAWINGS

The invention can be further understood by reference to the following examples, which are provided by way of illustration and are not meant to be limiting.

FIG. 1 is a frontal view a wearer in which a magnetic embodiment of the coupled magnetic system is placed on the upper right eyelid of the wearer. In this embodiment, the base component 1 is coated with a first magnetic element, while the band component 2 is comprised of a corresponding second magnetic element.

FIG. 2 is a frontal view of a coupled magnetic system in which the base component 2 has two magnetic elements (circles) spaced apart, while the band component has two corresponding second magnetic elements that correspond in distance to the first magnetic elements in the base component.

FIG. 3A is a side view of an embodiment of the coupled magnetic system in FIG. 2. The dotted cross axis describe the span (s) and height of curvature (h) of the band component as fitted to the band component.

FIG. 3B is a side view of an embodiment of the coupled magnetic system in FIG. 1 in which the base component 1 is coated with a first magnetic element. The thickness (t) of the base component can vary as described above, with a maximum thickness of 7 mm mm. The wearer's eyelashes are trapped between the base component and band component.

FIG. 4 is a black and white picture of a wearer's eyelashes trapped between the base component 1 and band component 2.

FIG. 5 is a picture of a prototype coupled magnetic system from FIG. 4.

FIG. 6 is a picture of a prototype coupled magnetic system wherein both band component 2 and base components 1 are made from a vinyl-coated flexible magnetic tape, wherein the band component 2 and base component 1 are polarized in the same direction to optimize attraction.

FIG. 7 is a depiction of a prototype coupled magnetic system on a measuring board in which the base component has two segments of double-adhesive nano tape adhered to its bottom surface.

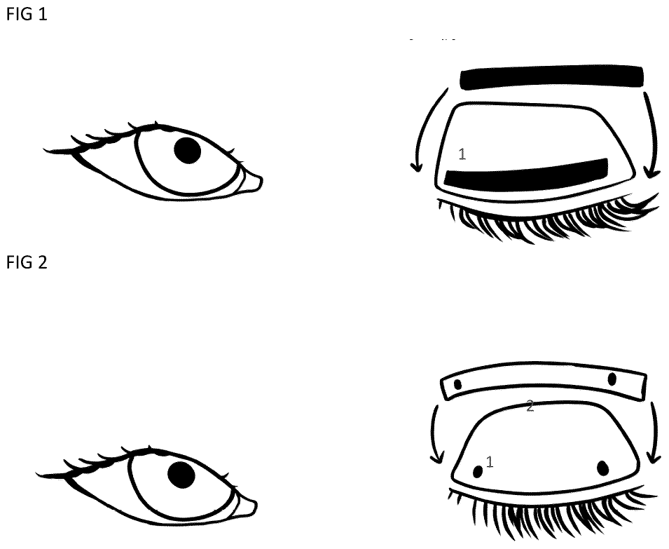

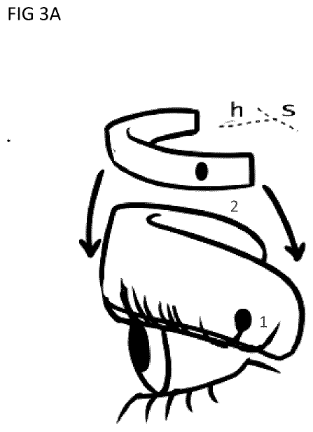

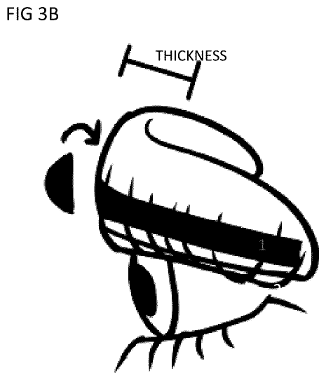

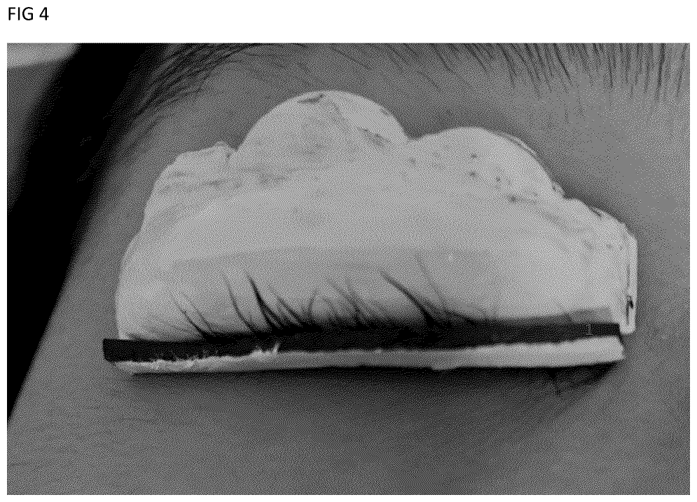

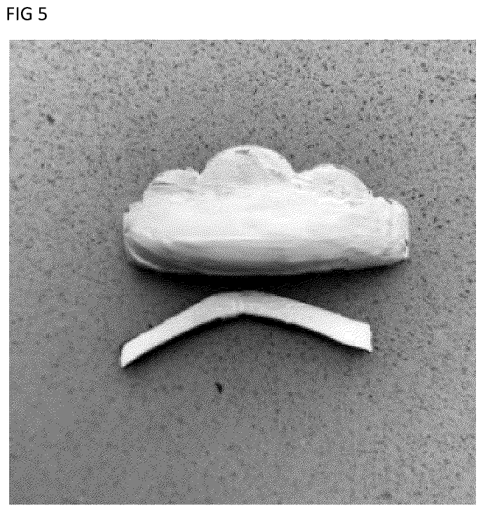

### EXAMPLE 1: STEP 1 PERMING SOLUTIONS

The following tables provide ingredients for perming solutions which comprise reducing agents such as thioglycolate.

Perming solution 1a:

| INGREDIENT | Weight % |
|---|---|
| water | 64.55 |
| | (to 100% balance) |
| Ethanolamine thioglycolate | 15.00 |
| Mineral oil | 5.00 |
| Cetearyl alcohol | 4.00 |
| Ethanolamine | 4.00 |
| Propylene glycol | 2.00 |
| Glyceryl stearate | 1.56 |
| PEG-100 stearate | 1.44 |
| Ceterimonium chloride | 1.00 |
| Petrolatum | 0.500 |
| Sorbitain sesquioleate | 0.500 |
| Hydrolyzed collagen | 0.15 |
| Etidronic acid | 0.20 |
| fragrance | 0.100 |

Perming solution 1b:

| INGREDIENT | Weight % |
|---|---|
| Water | 67.82 |
| | (to 100% balance) |
| Thioglycolic acid | 9.00 |
| Ethanolamine | 9.20 |
| Cetearyl alcohol | 3.00 |
| Urea | 1.50 |
| Polyquaternium-28 | 3.00 |
| Glyceryl stearate | 0.80 |
| Steartrimonium chloride | 2.00 |
| C12-15 pareth-10 | 2.00 |
| Glycerin | 1.00 |
| Fragrance | 0.50 |
| Disodium EDTA | .18 |

### EXAMPLE 2: SETTING SOLUTIONS

Perming solutions also include the step 2 setting solution, which is applied after the initial step 1 perming solution having the reducing agent, thioglycolate. The setting solution comprises but is not limited to an oxidizing agent such as hydrogen peroxide or sodium bromate. The following tables provide ingredients for the second step setting solutions.

Setting solution 2a:

| Ingredient | Weight % |
|---|---|
| Water | 84.50 |
| | (to 100% balance) |
| Sodium bromate | 8.00 |
| 1-hexadecanol | 3.50 |
| Steartrimonium chloride | 2.00 |
| Dipotassium glycyrrhizate | 1.50 |
| Disodium phosphate | 0.50 |

Setting solution 2b:

| Ingredient | Weight % |
|---|---|
| Water | 83.17979 |
| | (to 100% balance) |
| Hydrogen Peroxide | 5.80 |
| Cetearyl Alcohol | 4.00 |
| Mineral Oil | 3.00 |
| Sodium Laueth Sulfate | 1.00 |
| Behentrimoium Chloride | 0.80 |
| Glyceryl Stearate | 0.90 |
| Ceteth-20 | 0.80 |
| Disodium Phosphate | 0.20 |
| Phosphoric Acid | 0.12 |
| Phenacetin | 0.10 |
| CI 42090 brilliant blue | 0.00021 |
| Fragrance | 0.100 |

### EXAMPLE 3: COUPLED MAGNETIC SYSTEM FROM VINYL-COATED FLEXIBLE MAGNET

A coupled magnetic system can be prepared from vinyl-coated flexible magnets, which are a preferred low-cost option. One example of such a coupled magnetic system is prepared from a flexible magnet having the following technical requirements:
Grade F1
Magnetization: Axially Mganetized
Br: 1600 Gauss
BHmax: 0.60 MGOe
Hc: 1370 Oersteds
Max Operating Temperature: 212° F./100° C.
Density (lb/in3): 0.133

The flexible magnet is prepared from a dry mixture of ferrite powder and rubber polymer resin, which is mixed, calendered and ground, then formed by either extrusion or rollers and coated with white vinyl. Methods for manufacturing flexible magnets are well known in the art.

### EXAMPLE 4: COUPLED MAGNETIC SYSTEM WITH DOUBLE-ADHESIVE TAPE

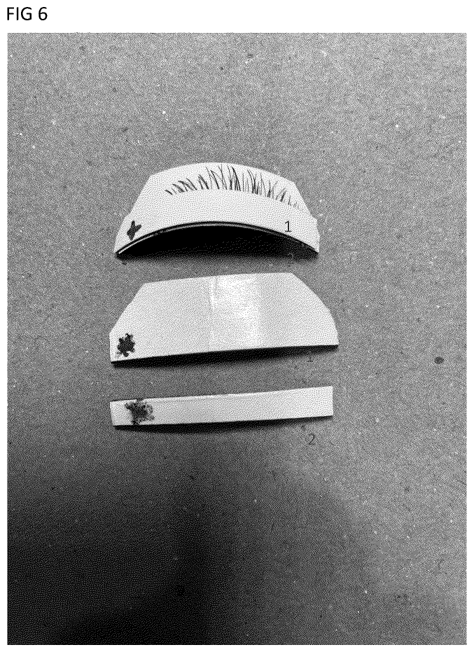

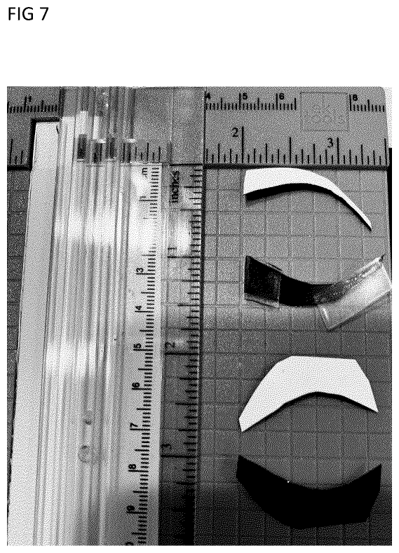

In another embodiment of the current invention, the base component and band components are made from magnetic strips coated on one side with white dry erase surface as shown in FIG. 6. The base component and band component are cut into magnetic strips in a roughly trapezoid shape that is similar to an eyelid for comfortable placement (see FIG. 7), with each being roughly about 0.7 in (17.8 mm) to about 2 in (50 mm) in length, or more preferably about 1 in (25 mm) to about 1.5 in (38 mm) in length.

A nano tape is further applied to the bottom surface of the base component for application and positioning to a wearer's eyelid (see FIG. 7). Examples of white magnetic strips include, but are not limited, to X-bet MAGNET and have a thickness varying from 0.01 in (0.25 mm) to 0.04 in (2 mm). In one embodiment, the preferred thickness for the magnetic strip is about 0.02 in (0.5 mm). The thickness of nano tape can vary from ranges of about 0.01 in (0.25 mm) to about 1.5 in to 38 mm with a preferred range of about 0.04 in (1 mm) to about 0.5 in (13 mm). Varying the thickness of the nano tape allows for control of the upturned eyelash curl, with lower thickness of about 0.04 in (1 mm) to about 0.08 in (2 mm) providing a more upright curl which is especially advantageous for shorter eyelashes. In one embodiment, the nano tape is roughly the same length as the base component. In an alternate preferred embodiment, the nano tape is applied only to the inner and outer corners of the base component, which each segment of nano tape being approximately 0.5 in (1 cm) to about 0.8 in (2 cm). For example, where a base component is roughly 4 cm long, in this alternate embodiment, the nano tape is divided into two segments and adhered to at least 0-1 and 3-4 centimeters of the base component (see FIG. 7). Positioning placement of the nano tape segments on only on the inner and outer corners of the base component allows the base component to adhere more closely to the contour of the wearer's eyelid since the rigidity of the magnetic strip will otherwise flatten (pop-up) away from the wearer's eyelid.

Nano tape as applied in the current invention has unexpected advantages; nano tape is (1) reusable, (2) performs well at room or high temperature, (3) adheres more closely to magnetic or plastic surfaces than to skin and are thus easily removed with the base component; (4) do not leave residue and (5) can be manufactured in varying thickness.

The coupled magnetic system with double-adhesive should ideally be less than 0.1 oz so as to be lightweight enough to stay placed on wearer's eyelid.

As shown in FIG. 6 and FIG. 7, the current invention comprises a kit for the application of solutions to a wearer's eyelashes wherein a coupled magnetic system is placeable on a wearer's eyelid, comprising:
a) the coupled magnetic system comprising a base component having a first magnetic element with double-adhesive tape (i.e. double adhesive nano tape) and a band component having a second magnetic element; and
b) a solution for application comprising a reducing agent, an oxidizing agent or dye-tint;
wherein the base component has a maximum thickness of 6 mm, and wherein the kit avoids glue.

This current invention comprises methods of using a coupled magnetic system with double-adhesive tape to apply solutions to eyelashes comprising the following steps:
(a) placement of a base component with double adhesive tape on its bottom surface on top of a wearer's eyelid;
(b) application of a solution to a wearer's eyelashes; and placement of a band component having a corresponding second magnetic element on top of the wearer's eyelashes such that the base component traps the eyelashes to the surface of the base component for a duration to allow the solution to effectively react with the wearer's eyelashes.

Embodiments of the applicator comprising a coupled magnetic system would have adequate gauss to hold the weight of the eyelash curler when the wearer or user is partially reclined, such as 200 gauss to 2000 gauss but not too strong so as to render separation of the applicator jaws difficult. An adequate gauss range for an applicator would be 200 gauss, 300 gauss, 400 gauss, 500 gauss, 600 gauss, 700 gauss, 800 gauss, 900 gauss, 1000 gauss, 1100 gauss, 1200 gauss, 1300 gauss, 1400 gauss, 1500 gauss, 1600 gauss, 1700 gauss, 1800 gauss, 1900 gauss, 2000 gauss or ranges within.

The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

We claim:

1. A kit for the application of solutions to a wearer's eyelashes wherein the kit comprises a coupled magnetic system placeable on a wearer's eyelid, comprising:
a) a base component having a first magnetic element with double-adhesive nano tape and a band component having a second magnetic element, and wherein the kit avoids glue.

2. The kit according to claim 1, wherein the base component has a maximum thickness of 6 mm.

3. The kit according to claim 1, wherein the base component has a span from about 25 cm to about 45 cm.

4. The kit according to claim 1 wherein the base component has a first magnetic element comprising iron.

5. The kit according to claim 4, wherein the band component comprises a corresponding second magnetic element comprised of iron ferrite powder.

6. The kit according to claim 1, wherein the coupled magnetic system with double-adhesive weighs less than 0.1 oz.

7. The kit according to claim 1, wherein the nano tape is divided into two segments.

8. The kit according to claim 7, wherein the nano tape segments is at least 1 cm.

9. The kit according to claim 1, wherein the kit further comprises a solution for application comprising a reducing agent, an oxidizing agent or dye-tint.

10. The kit according to claim 1, wherein the kit comprises a step 1 perming solution comprising a reducing agent.

11. The kit according to claim 5, wherein the reducing agent is thioglycolate.

12. The kit according to claim 5, wherein the kit comprises a step 2 setting solution comprising an oxidizing agent.

13. The kit according to claim 7, wherein the oxidizing agent is selected from the group consisting of hydrogen peroxide and sodium bromate.

14. The kit according to claim 1, wherein the kit further comprises a dye tint solution.

15. The kit according to claim 1, wherein the coupled magnetic system is further painted in a white color.

16. The kit according to claim, wherein the coupled magnetic system is prepared from ferrite powder and rubber polymer resin.

17. The kit according to claim 11, wherein the coupled magnetic system is further coated with white vinyl.

18. A method of using a coupled magnetic system to apply solutions to eyelashes comprising the following steps:
methods of using a coupled magnetic system with double-adhesive tape to apply solutions to eyelashes comprising the following steps:
(1) placement of a base component with double adhesive tape on its bottom surface on top of a wearer's eyelid;
(2) application of a solution to a wearer's eyelashes; and placement of a band component having a corresponding second magnetic element on top of the wearer's eyelashes such that the base component traps the eyelashes to the surface of the base component for a duration to allow the solution to effectively react with the wearer's eyelashes,
wherein the method does not comprise the step of applying a liquid adhesive directly to the eyelashes.

19. The method according to claim 14, wherein the solution is a perming solution.

20. The method according to claim 14, wherein the solution is a dye-tint solution.
